(19) United States
(12) Patent Application Publication
(10) Pub. No.: US 2022/0370445 A1
(43) Pub. Date: Nov. 24, 2022

(54) METHODS FOR ADMINISTERING (R)-N-[4-(1,4,5,6-TETRAHYDRO-6-OXO-3-PYRIDAZINYL)PHENYL]ACETAMIDE

(71) Applicant: ORION CORPORATION, Eapoo (FI)

(72) Inventors: Jouko LEVIJOKI, Helsinki (FI); Sari PAPPINEN, Raisio (FI); Lasse SALORANTA, Sauvo (FI)

(21) Appl. No.: 17/623,925

(22) PCT Filed: Jul. 30, 2020
(86) PCT No.: PCT/FI2020/050475

§ 371 Date: Dec. 30, 2021

(30) Foreign Application Priority Data
Jul. 1, 2019 (FI) 20195595

(51) Int. Cl. A61K 31/50; A61K 9/00; A61K 31/55

(52) U.S. Cl. CPC A61K 31/50; A61K 9/006; A61K 9/0043; A61K 9/0048; A61K 9/0075; A61K 31/55

(57) ABSTRACT

The invention relates to ocular, intranasal, oromucosal or pulmonary administration of (R)—N-[4-(1,4,5,6-tetrahydro-4-methyl-6-oxo-3-pyridazinyl)phenyl]acetamide (I) for the treatment of diseases and conditions where, for example, inotropic, vasodilatory or calcium sensitizing effects are desired. The invention also relates to pharmaceutical dosage forms adapted for ocular, intranasal, oromucosal or pulmonary administration comprising (R)—N-[4-(1,4,5,6-tetrahydro-4-methyl-6-oxo-3-pyridazinyl)phenyl]acetamide (I) as an active ingredient.

### TECHNICAL FIELD

The present invention relates to ocular, intranasal, oromucosal or pulmonary administration of (R)—N-[4-(1,4,5,6-tetrahydro-6-oxo-3-pyridazinyl)phenyl]acetamide (I) for the treatment of diseases and conditions where, for example, inotropic, vasodilatory and calcium sensitizing effects are desired, such as, for example, acute and chronic heart failure, renal failure, pulmonary hypertension, pre- or perioperative use during heart surgery, prevention of stroke or transient ischemic attack, subarachnoid haemorrhage, sepsis and amyotrophic lateral sclerosis. The present invention also relates to pharmaceutical dosage forms comprising (R)—N-[4-(1,4,5,6-tetrahydro-6-oxo-3-pyridazinyl)phenyl]acetamide (I) as an active ingredient.

### BACKGROUND OF THE INVENTION

### (R)—N-[4-(1,4,5,6-tetrahydro-4-methyl-6-oxo-3-pyridazinyl)phenyl]acetamide (I)

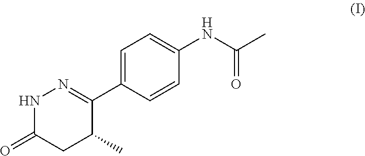 [SMILES: CC(=O)Nc1ccc(C2=NNC(=O)C[C@H]2C)cc1]

is known to be present as an active metabolite in humans following administration of levosimendan, an agent currently used for the treatment of acutely decompensated chronic heart failure. Levosimendan and its active metabolite (I) have similar hemodynamic effects. Therefore, compound (I) is believed to be useful in the treatment of conditions and diseases which respond favourably to levosimendan treatment. The use of levosimendan and its active metabolite (I) for the treatment of various conditions and diseases has been described e.g. in WO 99/66932, WO 2003/004035, WO 2004/060375, WO 2005/107756 and WO 2016/059287.

### SUMMARY OF THE INVENTION

It has been found that (R)—N-[4-(1,4,5,6-tetrahydro-4-methyl-6-oxo-3-pyridazinyl)phenyl]acetamide (I) is particularly suitable for ocular, intranasal, oromucosal or pulmonary administration resulting in rapid systemic absorption, good bioavailability, lack of significant irritation and extended elimination phase. The pharmaceutical compositions for ocular, intranasal, oromucosal and pulmonary administration provide rapid therapeutic effect by non-invasive administration route and are particularly suitable for patients who experience problems in swallowing oral dosage forms such as tablets or capsules. It was also found that the sinus node I_{f} channel inhibitor ivabradine or its active metabolite N-desmethyl ivabradine or a pharmaceutically acceptable salt thereof can be administered in combination with (R)—N-[4-(1,4,5,6-tetrahydro-4-methyl-6-oxo-3-pyridazinyl)phenyl]acetamide (I), e.g. in ocular, intranasal, oromucosal and pulmonary administration, to offset the heart rate increase which is associated with (R)—N-[4-(1,4,5,6-tetrahydro-4-methyl-6-oxo-3-pyridazinyl)phenyl]acetamide (I) administration particularly at higher doses.

Thus, according to one embodiment of the invention, the present invention provides a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) to a patient by ocular, intranasal, oromucosal or pulmonary administration.

According to another embodiment of the invention, the present invention provides a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) to a patient by ocular administration.

According to another embodiment of the invention, the present invention provides a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) to a patient by intranasal administration.

According to still another embodiment of the invention, the present invention provides a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) to a patient by oromucosal administration.

According to still another embodiment of the invention, the present invention provides a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) to a patient by pulmonary administration.

According to another embodiment of the invention, the present invention provides (R)—N-[4-(1,4,5,6-tetrahydro-4-methyl-6-oxo-3-pyridazinyl)phenyl] acetamide (I) for use in the method of ocular, intranasal, oromucosal or pulmonary administration of (R)—N-[4-(1,4,5,6-tetrahydro-4-methyl-6-oxo-3-pyridazinyl)phenyl] acetamide (I).

According to another embodiment of the invention, the present invention provides the use of (R)—N-[4-(1,4,5,6-tetrahydro-4-methyl-6-oxo-3-pyridazinyl)phenyl]acetamide (I) in the manufacture of a medicament for ocular, intranasal, oromucosal or pulmonary administration of (R)—N-[4-(1,4,5,6-tetrahydro-4-methyl-6-oxo-3-pyridazinyl)phenyl]acetamide (I).

According to another embodiment of the invention, the present invention provides a pharmaceutical composition adapted for use in ocular, intranasal, oromucosal or pulmonary administration comprising (R)—N-[4-(1,4,5,6-tetrahydro-4-methyl-6-oxo-3-pyridazinyl)phenyl]acetamide (I) as an active ingredient.

According to another embodiment of the invention, the present invention provides an eye drop composition comprising (R)—N-[4-(1,4,5,6-tetrahydro-4-methyl-6-oxo-3-pyridazinyl)phenyl]acetamide (I) as an active ingredient.

According to another embodiment of the invention, the present invention provides an intranasal composition comprising (R)—N-[4-(1,4,5,6-tetrahydro-4-methyl-6-oxo-3-pyridazinyl)phenyl]acetamide (I) as an active ingredient.

According to another embodiment of the invention, the present invention provides an oromucosal composition comprising (R)—N-[4-(1,4,5,6-tetrahydro-4-methyl-6-oxo-3-pyridazinyl)phenyl]acetamide (I) as an active ingredient.

According to another embodiment of the invention, the present invention provides an inhalation composition comprising (R)—N-[4-(1,4,5,6-tetrahydro-4-methyl-6-oxo-3-pyridazinyl)phenyl]acetamide (I) as an active ingredient.

According to another embodiment of the invention, the present invention provides an inhaler or nebulizer device comprising a composition of (R)—N-[4-(1,4,5,6-tetrahydro-4-methyl-6-oxo-3-pyridazinyl)phenyl]acetamide (I) adapted for pulmonary administration.

According to still one embodiment of the invention, the present invention provides an eye drop, intranasal, oromucosal or inhalation composition comprising (R)—N-[4-(1,4,5,6-tetrahydro-4-methyl-6-oxo-3-pyridazinyl)phenyl]acetamide (I) as a sole active ingredient.

Moreover, according to one embodiment of the invention, the present invention provides a method for administration of a composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof to a patient in need thereof comprising administering said composition to a patient by ocular, intranasal, oromucosal or pulmonary administration.

According to another embodiment of the invention, the present invention provides a method for administration of a composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof to a patient in need thereof comprising administering said composition to a patient by ocular administration.

According to another embodiment of the invention, the present invention provides a method for administration of a composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof to a patient in need thereof comprising administering said composition to a patient by intranasal administration.

According to still another embodiment of the invention, the present invention provides a method for administration of a composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof to a patient in need thereof comprising administering said composition to a patient by oromucosal administration.

According to still another embodiment of the invention, the present invention provides a method for administration of a composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof to a patient in need thereof comprising administering said composition to a patient by pulmonary administration.

According to another embodiment of the invention, the present invention provides a composition comprising (R)—N-[4-(1,4,5,6-tetrahydro-4-methyl-6-oxo-3-pyridazinyl)-phenyl]acetamide (I) in combination with ivabradine or N-desmethyl ivabradine or a pharmaceutically acceptable salt thereof for use in the method of administering the composition by ocular, intranasal, oromucosal or pulmonary administration.

According to another embodiment of the invention, the present invention provides the use of (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof, in the manufacture of a medicament for ocular, intranasal, oromucosal or pulmonary administration.

According to another embodiment of the invention, the present invention provides a pharmaceutical composition adapted for use in ocular, intranasal, oromucosal or pulmonary administration comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.

According to another embodiment of the invention, the present invention provides an eye drop composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.

According to another embodiment of the invention, the present invention provides an intranasal composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.

According to another embodiment of the invention, the present invention provides an oromucosal composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.

According to another embodiment of the invention, the present invention provides an inhalation composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.

According to another embodiment of the invention, the present invention provides an inhaler or nebulizer device comprising a composition of (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof, adapted for pulmonary administration.

According to still another embodiment of the invention, the present invention provides a pharmaceutical composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.

### BRIEF DESCRIPTION OF THE DRAWINGS

FIG. 1 shows plasma concentrations of (R)—N-[4-(1,4,5,6-tetrahydro-4-methyl-6-oxo-3-pyridazinyl)phenyl]acetamide (I) in systemic circulation after ocular (4 μg/kg), intranasal (4 μg/kg) or oromucosal dosing (5 μg/kg) compared to 4 μg/kg intravenous (i.v.) administration in Beagle dogs (n=6).

FIG. 2 shows plasma concentrations of (R)—N-[4-(1,4,5,6-tetrahydro-4-methyl-6-oxo-3-pyridazinyl)phenyl]acetamide (I) in systemic circulation after oropharyngeal dosing (10 or 20 μg/kg) compared to 10 μg/kg intravenous (i.v.) administration in Male Sprague Dawley rats.

FIG. 3 shows changes in the heart rate in Beagle dogs after oromucosal administration of (R)—N-[4-(1,4,5,6-tetrahydro-4-methyl-6-oxo-3-pyridazinyl)phenyl]acetamide (I) (Compound A, 18.5 μg/kg) alone or together with ivabradine hydrochloride (Compound B, 1 mg/kg).

### DETAILED DESCRIPTION OF THE INVENTION

The term “administering to the eye” or “ocular administration”, as used herein, refers to applying topically to the eye and surrounding tissues, particularly to the inner surface of the eye and the inner surface of the eyelids (including e.g. cornea, conjunctiva and sclera). The term includes, for example, instillation administration, administration into conjunctival sac and conjunctival administration.

The term “administering to the nasal mucosa” or “intranasal administration”, as used herein, refers to applying topically to any portion of the nasal epithelium.

The term “administering to the oral mucosa” or “oromucosal administration”, as used herein, refers to applying topically to mucosa of the mouth, particularly to buccal mucosa, the sublingual mucosa, the gum, or the inner lip.

The term “eye drop composition”, as used herein, refers to a liquid or semisolid pharmaceutical composition adapted to administration to the eye. Typical example of an eye drop composition is an ophthalmic aqueous solution to be administered dropwise to the eye.

The term “pulmonary administration”, as used herein, refers to administration to the lungs of the patient, particularly by inhalation of an inhalable composition such as dry powder, an aerosol or a mist of suspension or solution.

The term “(R)—N-[4-(1,4,5,6-tetrahydro-4-methyl-6-oxo-3-pyridazinyl)phenyl]acetamide (I)” or “compound (I)” refers to compound which is (R)—N-[4-(1,4,5,6-tetrahydro-4-methyl-6-oxo-3-pyridazinyl)phenyl]acetamide and to pharmaceutically acceptable salts and solvates thereof.

The term “patient” refers to a mammal including human.

The present invention relates to a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)-phenyl]acetamide (I) to a patient by ocular, intranasal, oromucosal or pulmonary administration.

The present invention also relates to a method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof, to a patient. In one preferred embodiment of the invention, the combination is administered to a patient by ocular, intranasal, oromucosal or pulmonary administration.

Compound (I) is administered to mammals, including humans, generally in an amount ranging from about 0.1 μg/kg to about 50 μg/kg, preferably from about 0.2 μg/kg to about 20 μg/kg, typically from about 0.5 μg/kg to about 10 μg/kg, given once a day or divided into several doses a day. Compound (I) may also be administered periodically, e.g. weekly or biweekly, depending on the patient's needs. The actual amount of compound (I) to be administered may depend on numerous factors, such as the species, age and weight of the subject to be treated, the condition to be treated, the administration route and the type of the composition. Suitable plasma concentrations of compound (I) are generally within the range from about 0.1 ng/ml to about 10.0 ng/ml, or from about 0.1 ng/ml to about 5.0 ng/ml, preferably from about 0.5 ng/ml to about 2.5 ng/ml, for example from about 1.0 ng/ml to about 1.5 ng/ml. The elimination half-life of compound (I) in man is rather long, about 72 h. Therefore, a periodical treatment, e.g. weekly, may be satisfactory.

Ivabradine or N-desmethyl ivabradine or a pharmaceutically acceptable salt thereof, for example ivabradine hydrochloride, is administered to mammals, including humans, generally in an amount ranging from about 0.01 mg/kg to about 1.5 mg/kg, preferably from about 0.02 mg/kg to about 1 mg/kg, typically from about 0.05 mg/kg to about 0.5 mg/kg, given once a day or divided into several doses a day.

Compound (I), optionally in combination with ivabradine or N-desmethyl ivabradine or a pharmaceutically acceptable salt thereof, can be formulated into a dosage form adapted for administration to the eye by combining the drug substance or drug substances with conventional pharmaceutical diluents and carriers commonly used in eye drop compositions. However, it was found that compound (I), as well as ivabradine or N-desmethyl ivabradine or a pharmaceutically acceptable salt thereof, are readily adsorbed into the plasma from a plain aqueous eye drop solution, such that any penetration enhancing agents are not needed. The eye drop composition useful in the method of the invention may be, for example, in a liquid or semisolid form such as in the form of a solution, emulsion, suspension or gel.

According to one embodiment, a single dose of the eye drop composition, given to one eye or divided to both eyes, is able to provide a plasma concentration of compound (I) of at least about 0.5 ng/ml, preferably at least about 1 ng/ml, more preferably from about 1 to about 5 ng/ml, for example from about 1 to about 2 ng/ml, within about 15 minutes, preferably within about 10 minutes, for example within about 5 minutes, from the time of administration.

Preferably, the eye drop composition is in the form of an aqueous solution adapted for administration to the eye of the subject. The concentration of compound (I) in the eye drop composition, e.g. in the aqueous solution composition, is generally within the range of about 0.001 to about 0.5%, typically from about 0.005 to about 0.1%, preferably from about 0.01 to about 0.07%, for example from about 0.03 to about 0.06%, per weight of the composition. If ivabradine or N-desmethyl ivabradine or a pharmaceutically acceptable salt thereof is also used, its concentration in the eye drop composition is generally within the range of about 0.01 to about 5%, typically from about 0.05 to about 2%, for example from about 0.1 to about 1%, per weight of the composition.

According to one embodiment, the eye drop composition comprises 0.001-0.5%, preferably 0.005-0.1%, more preferably from about 0.01 to about 0.07%, for example from about 0.03 to about 0.06%, per weight of the composition, of compound (I), and at least 90%, preferably at least 95%, for example at least 97.5%, per weight of the composition, of sterile water.

According to another embodiment, the eye drop composition comprises 0.001-0.5%, preferably 0.005-0.1%, more preferably from about 0.01 to about 0.07%, for example from about 0.03 to about 0.06%, per weight of the composition, of compound (I), about 0.01-5%, preferably 0.05-1%, more preferably from about 0.05 to about 0.35%, for example from about 0.1 to about 0.7%, per weight of the composition, of ivabradine or N-desmethyl ivabradine or a pharmaceutically acceptable salt thereof, and at least 90%, preferably at least 95%, for example at least 97.5%, per weight of the composition, of sterile water.

The eye drop composition may additionally comprise a tonicity adjusting agent such as sodium chloride or mannitol, pH adjusting agents or buffering agents such as sodium hydroxide, hydrochloric acid, citric acid/sodium citrate, tartaric acid, fumaric acid, antioxidants such as butylated hydroxyanisole (BHA) or butylated hydroxytoluene (BHT), chelating agents such as edetate disodium, thickening agents such as polyvinylpyrrolidone (povidone), polyvinyl alcohol, polyethylene glycol, polyacrylic acid or a cellulose derivative such as sodium carboxymethylcellulose, preservatives such as benzalkonium chloride, parabens or benzyl alcohol, chelating agents such as disodium edetate and other ingredients commonly used in the preparation of eye drop compositions.

Suitably, the eye drop composition comprises 0.5-2%, per weight of the composition, of a tonicity adjusting agent such as sodium chloride. The osmolality of the eye drop composition is suitably adjusted to 200-600 mOsm/kg, preferably to about 300 mOsm/kg.

The eye drop composition may also suitably comprise 0.1-5%, preferably 0.5-3%, per weight of the composition, of a thickening agent, for example polyvinyl-pyrrolidone.

The pH of the eye drop composition is generally within the range of from about 3 to about 8.5, preferably from about 4.0 to about 7.0, more preferably from about 4.5 to about 6.5.

The eye drop formulation can be prepared e.g. by dissolving the active ingredient(s) and excipients to the vehicle, for example water, followed by pH adjustment, if necessary, and sterile filtering.

The eye drop composition is preferably given to one eye or divided to both eyes of the subject from a prefilled bottle, ampoule or pipette in a volume ranging typically from 0.01 to about 0.3 nil, more preferably from about 0.015 to about 0.2 nil, for example from 0.02 to 0.15 ml, of the eye drop composition.

The eye drop composition comprises suitably from about 0.1 mg/ml to about 1 mg/ml, preferably from about 0.2 mg/ml to about 0.7 mg/ml, for example from about 0.3 mg/ml to about 0.6 mg/ml, of compound (I). If ivabradine or N-desmethyl ivabradine or a pharmaceutically acceptable salt thereof is also used, the eye drop composition comprises suitably from about 0.5 mg/ml to about 20 mg/ml, preferably from about 1 mg/ml to about 15 mg/ml, for example from about 2 mg/ml to about 10 mg/ml, of ivabradine or N-desmethyl ivabradine or a pharmaceutically acceptable salt thereof.

An unit dose of the eye drop composition for humans comprises an amount of compound (I) which is generally in the range from about 1 μg to about 1000 μg, preferably from 10 μg to 800 μg, for example from 25 μg to 600 μg, to be given to one eye or divided to both eyes. If ivabradine or N-desmethyl ivabradine or a pharmaceutically acceptable salt thereof is also used, its amount in the unit dose of the eye drop composition is generally in the range from about 0.01 mg to about 50 mg, preferably from 0.1 mg to 30 mg, for example from 0.25 mg to 20 mg, to be given to one eye or divided to both eyes.

Preferably, the eye drop composition is packaged in an applicator, e.g. a squeezable prefilled single-use bottle, ampoule or pipette capable of dosing fixed volumes of the eye drop composition. The squeezable bottle, ampoule or pipette is preferably prepared form polymer material, such as LDPE. Suitably, the volume of the suitable bottle, ampoule or pipette ranges from about 0.5 to 5 ml. For example, about 0.5 to about 2 ml of the eye drop composition can be filled into single use blow fill seal (BFS) LDPE ampoules having volume of 0.5 nil, 1 ml or 2 nil.

Compound (I), optionally in combination with ivabradine or N-desmethyl ivabradine or a pharmaceutically acceptable salt thereof, can also be formulated into a dosage form adapted for intranasal administration by combining the drug substance or drug substances with conventional pharmaceutical diluents and carriers commonly used in intranasal compositions. However, it was found that compound (I) as well as ivabradine or N-desmethyl ivabradine or a pharmaceutically acceptable salt thereof, are readily adsorbed into the plasma from a plain aqueous intranasal solution, such that any penetration enhancing agents are not needed. The intranasal composition is suitably an intranasal drop or spray (fine mist) in a liquid form such as, for example, a solution, emulsion or suspension.

According to one embodiment, a single dose of the intranasal composition, given to one nostril or divided to both nostrils, is able to provide a plasma concentration of compound (I) of at least about 0.5 ng/ml, preferably at least about 1 ng/ml, more preferably from about 1 to about 5 ng/ml, for example from about 1 to about 2 ng/ml, within about 15 minutes, preferably within about 10 minutes, for example within about 5 minutes, from the time of administration.

Preferably, the intranasal composition is in the form of an aqueous solution adapted for administration to the nasal cavities of the subject. The concentration of compound (I) in the intranasal composition, e.g. in the aqueous solution composition, is generally within the range of about 0.001 to about 0.5%, typically from about 0.002 to about 0.1%, preferably from about 0.005 to about 0.07%, for example from about 0.01 to about 0.05%, per weight of the composition. If ivabradine or N-desmethyl ivabradine or a pharmaceutically acceptable salt thereof is also used, its concentration in the intranasal composition is generally within the range of about 0.005 to about 1%, typically from about 0.01 to about 0.5%, for example from about 0.02 to about 0.2%, per weight of the composition.

According to one embodiment, the intranasal composition comprises from about 0.001 to about 0.5%, preferably from about 0.002 to about 0.1%, more preferably from about 0.005 to about 0.07%, for example from about 0.01 to about 0.05%, per weight of the composition, of compound (I), and at least 90%, preferably at least 95%, for example at least 97.5%, per weight of the composition, of sterile water.

According to another embodiment, the intranasal composition comprises from about 0.001 to about 0.5%, preferably from about 0.002 to about 0.1%, more preferably from about 0.005 to about 0.07%, for example from about 0.01 to about 0.05%, per weight of the composition, of compound (I), from about 0.005 to about 1%, preferably from about 0.01 to about 0.5%, for example from about 0.02 to about 0.2%, per weight of the composition, of ivabradine or N-desmethyl ivabradine or a pharmaceutically acceptable salt thereof, and at least 90%, preferably at least 95%, for example at least 97.5%, per weight of the composition, of sterile water.

The intranasal composition may additionally comprise a tonicity adjusting agent such as sodium chloride or mannitol, pH adjusting agents or buffering agents such as sodium hydroxide, hydrochloric acid, citric acid/sodium citrate, tartaric acid, fumaric acid, antioxidants such as butylated hydroxyanisole (BHA) or butylated hydroxytoluene (BHT), chelating agents such as edetate disodium, thickening agents such as polyvinylpyrrolidone (povidone), polyvinyl alcohol, polyethylene glycol, polyacrylic acid or a cellulose derivative such as sodium carboxymethylcellulose, chelating agents such as disodium edetate, preservatives such as benzalkonium chloride, parabens or benzyl alcohol, humectants such as cetyl palmitate, glycerol (glycerin), lanolin alcohol or lanolin, flavouring agents and other ingredients commonly used in the preparation of intranasal compositions.

Suitably, the intranasal composition comprises 0.5-2%, per weight of the composition, of a tonicity adjusting agent such as sodium chloride. The osmolality of the intranasal composition is suitably adjusted to 200-600 mOsm/kg, preferably to about 300 mOsm/kg.

The intranasal composition may also suitably comprise 0.1-5%, preferably 0.5-3%, per weight of the composition, of a thickening agent, for example polyvinyl-pyrrolidone.

The pH of the intranasal composition is generally within the range of from about 3 to about 8.5, preferably from about 4.0 to about 7.0, more preferably from about 4.5 to about 6.5.

The intranasal formulation can be prepared e.g. by dissolving the active ingredient and excipients to the vehicle, for example water, followed by pH adjustment, if necessary, and sterile filtering.

The intranasal composition is preferably given to one nostril or divided to both nostrils of the subject from a prefilled spray pump, ampoule or pipette in a volume ranging typically from about 0.01 to about 1 nil, more preferably from about 0.02 to about 0.75 nil, for example from about 0.05 to about 0.5 nil, of the intranasal composition.

The intranasal composition comprises suitably from about 0.02 mg/ml to about 1 mg/ml, preferably from about 0.05 mg/ml to about 0.7 mg/ml, for example from about 0.1 mg/ml to about 0.5 mg/ml, of compound (I). If ivabradine or N-desmethyl ivabradine or a pharmaceutically acceptable salt thereof is also used, the intranasal composition comprises suitably from about 0.1 mg/ml to about 5 mg/ml, preferably from about 0.25 mg/ml to about 3 mg/ml, for example from about 0.5 mg/ml to about 2 mg/ml, of ivabradine or N-desmethyl ivabradine or a pharmaceutically acceptable salt thereof.

An unit dose of the intranasal composition for humans comprises an amount of compound (I) which is generally in the range from about 1 μg to about 1000 μg, preferably from 10 μg to 800 μg, for example from 25 μg to 600 μg, If ivabradine or N-desmethyl ivabradine or a pharmaceutically acceptable salt thereof is also used, its amount in the unit dose of the intranasal composition is generally in the range from about 0.01 mg to about 50 mg, preferably from 0.1 mg to 30 mg, for example from 0.25 mg to 20 mg,

Preferably, the intranasal composition is packaged in an applicator, e.g. a nasal spray device capable of dosing a fixed volume of the intranasal composition. Nasal spray device typically comprises a bottle into which the liquid intranasal composition is placed, and an actuator which, when actuated, forces a spray plume of the composition out of the spray bottle. Alternatively, a squeezable prefilled single-use bottle, ampoule or pipette capable of dosing fixed volumes of the intranasal composition can be used. The applicator is preferably prepared form polymer material, such as LDPE. Suitably, the volume of spray bottle ranges from about 0.5 to 5 nil. For example, about 0.5 to about 2 ml of the intranasal composition can be filled into a spray bottle or squeezable bottle.

Compound (I), optionally in combination with ivabradine or N-desmethyl ivabradine or a pharmaceutically acceptable salt thereof, can also be formulated into a dosage form adapted for oromucosal administration. Such dosage forms include, for example, buccal, gingival or sublingual tablets, films or gels that dissolve or disintegrate, delivering drug into the mouth of the patient. Oromucosal composition can also be in the form of a solution, dispersion or emulsion which is sprayed or dropped to the mouth of the patient in such a small volume, which does not provoke a swallowing reflex. Oromucosal composition may also be mucoadhesive. Upon contact with intact mucous membrane of the mouth such mucoadhesive composition adheres to said mucous membrane for a sufficient time period to induce the desired therapeutic effect. Mucoadhesive composition may comprise a mucoadhesive agent.

Suitable mucoadhesive agents include those well known in the art such as polyacrylic acids, preferably having the molecular weight between from about 450,000 to about 4,000,000, e.g. Carbopol™ 934P; sodium carboxymethylcellulose (NaCMC); hydroxypropylmethyl cellulose (HPMC), e.g. Methocel™ K100; and hydroxypropyl cellulose.

The oromucosal composition can also be in the form of a patch or device that contains the drug and adheres to a mucosal surface. Suitable transmucosal patches are described for example in WO 93/23011. A suitable patch may comprise a backing. The backing can be any flexible film that prevents bulk fluid flow and provides a barrier for loss of the drug from the patch. The backing can be any of the conventional materials such as polyethylene, ethyl-vinyl acetate copolymer or polyurethane. In a patch involving a matrix which is not itself a mucoadhesive, the drug-containing matrix can be coupled with a mucoadhesive component (such as a mucoadhesive described above) to retain the patch on the oromucosal surface. Suitable configurations include a patch or device wherein the matrix has a smaller periphery than the backing layer such that a portion of the backing layer extends outward from the periphery of the matrix. A mucoadhesive layer covers the outward extending portion of the backing layer such that the underside of the backing layer carries a layer of mucoadhesive around its periphery. The backing and the peripheral ring of mucoadhesive taken together form a reservoir which contains a drug-containing matrix (e.g. a tablet, gel, or powder). It may be desirable to incorporate a barrier element between the matrix and the mucoadhesive in order to isolate the mucoadhesive from the matrix. The barrier element is preferably substantially impermeable to water and to the mucosal fluids that will be present at intended site of adhesion. A patch or device having such barrier element can be hydrated only through a surface that is in contact with the mucosa. Such patches can be prepared by general methods well known to those skilled in the art. Oromucosal compositions can contain pharmaceutical ingredients, such as fillers, lubricants, disintegrants, solubilizing vehicles, flavours or dyes. Oromucosal compositions may incorporate mucous membrane penetration enhancers such as anionic, cationic or non-ionic surfactants (e.g. sodium lauryl sulphate, sodium dodecyl sulphate, polysorbate 80, glyceryl monolaurate) and related compounds.

According to one embodiment, a single dose of the oromucosal composition, given to oral mucosa of the patient, is able to provide a plasma concentration of compound (I) of at least about 0.5 ng/ml, preferably at least about 1 ng/ml, more preferably from about 1 to about 5 ng/ml, for example from about 1 to about 2 ng/ml, within about 15 minutes, preferably within about 10 minutes, for example within about 5 minutes, from the time of administration.

The concentration of compound (I) in the oromucosal composition, e.g. in the oromucosal gel composition, is generally within the range of about 0.001 to about 1%, typically from about 0.002 to about 0.5%, preferably from about 0.005 to about 0.1%, for example from about 0.01 to about 0.05%, per weight of the composition. If ivabradine or N-desmethyl ivabradine or a pharmaceutically acceptable salt thereof is also used, its concentration in the oromucosal composition is generally within the range of about 0.01 to about 10%, typically from about 0.02 to about 5%, for example from about 0.05 to about 2%, per weight of the composition.

According to one embodiment of the invention, the oromucosal composition is a semisolid oromucosal gel. Such composition can be prepared by combining the drug substance with conventional pharmaceutical diluents and carriers commonly used in semisolid gel formulations. The semisolid oromucosal gel composition should be viscous enough for being able to remain on the oral mucosa, e.g. gingival mucosa, of the patient. However, the viscosity should not be so high that the composition could be easily swallowed. Preferably, the semisolid material should have a viscosity from about 500 to about 200,000 mPas, preferably from about 1,000 to about 100,000 mPas, more preferably from about 5,000 to about 50,000 mPas, for example from about 8,000 to about 30,000 mPas. According to one embodiment, the semisolid material has a viscosity from about 3000 mPas to about 50,000 mPas, particularly from about 5,000 mPas to about 20,000 mPas. The gel composition is suitably applied oromucosally in a small volume using a suitable applicator such as a syringe or the like.

According to one embodiment, the oromucosal gel formulation comprises compound (I), optionally ivabradine or N-desmethyl ivabradine or a pharmaceutically acceptable salt thereof, a gelling agent, water-miscible organic co-solvent and water. The formulation may also comprise a transmucosal penetration enhancer.

According to another embodiment, the oromucosal gel formulation comprises, per weight of the composition, from about 0.001 to about 0.5% of compound (I), from about 0.3 to about 40% of a gelling agent; from about 0.2 to about 15% of a transmucosal penetration enhancer; from about 5 to about 50% of a water-miscible organic co-solvent; and 30-80% of water.

According to another embodiment, the oromucosal gel formulation comprises, per weight of the composition, from about 0.001 to about 0.5% of compound (I), from about 0.02 to about 5% of ivabradine or N-desmethyl ivabradine or a pharmaceutically acceptable salt thereof, from about 0.3 to about 40% of a gelling agent; from about 0.2 to about 15% of a transmucosal penetration enhancer; from about 5 to about 50% of a water-miscible organic co-solvent; and 30-80% of water.

The gelling agent may be any suitable hydrophilic gel forming polymer. Suitable the gelling agents include cellulose derivatives such as hydroxypropyl cellulose or hydroxyethyl cellulose, polyacrylic acids such as carboxyvinyl polymers (carbomers) and polyoxyethylene/polyoxypropylene copolymers.

Water-miscible organic co-solvents suitable for use in the oromucosal gel compositions include polyalcohols or glycols such as propylene glycol, butylene glycol, ethylene glycol, preferably propylene glycol or C₂-C₄ alkanols such as ethanol, isopropanol, n-propanol or butanol; or combinations thereof. Preferred are non-volatile organic co-solvents, particularly propylene glycol.

Suitable penetration enhancers include anionic, cationic or non-ionic surfactants such as, for example, sodium lauryl sulphate, sodium dodecyl sulphate, polysorbate 80 and glyceryl mono laurate.

Compound (I), optionally in combination with ivabradine or N-desmethyl ivabradine or a pharmaceutically acceptable salt thereof, can also be formulated into a dosage form adapted for pulmonary administration, such as an inhalation composition to be inhaled by the patient. The inhalation composition can be in the form of a dry powder inhalation composition, a pressurized aerosol inhalation composition or a nebulized inhalation composition (e.g. aqueous suspension or solution). A dry powder inhalation composition is the preferred form.

The concentration of compound (I) in the inhalation composition is generally within the range of about 0.001 to about 10%, typically from about 0.002 to about 5%, preferably from about 0.005 to about 1%, for example from about 0.01 to about 0.5%, per weight of the composition. If ivabradine or N-desmethyl ivabradine or a pharmaceutically acceptable salt thereof is also used, its concentration in the inhalation composition is generally within the range of about 0.01 to about 10%, typically from about 0.02 to about 5%, preferably from about 0.05 to about 3%, for example from about 0.1 to about 1%, per weight of the composition.

To ascertain that the medicament particles reach the target site in the lungs, the active ingredient(s) should be in micronized form, i.e. having particle size lower than about 10 μm, for example in the range from about 0.5 to about 10 μm, particularly in the range from about 1 to about 6 μm, such as to be able to deposit target areas in the lungs. Conventional methods, such as milling, can be used to provide the active ingredient(s) in micronized form.

The inhalation composition typically comprises compound (I), and optionally ivabradine or N-desmethyl ivabradine or a pharmaceutically acceptable salt thereof, as particles having volume median diameter in the range from about 0.5 μm to about 10 μm, preferably from about 1 μm to about 5 μm. It is particularly preferred that at least 90 w-%, preferably 95 w-%, of particles of compound (I) is in the range from about 0.5 μm to about 10 μm, preferably in the range from about 0.5 μm to about 5 μm.

The particle size of particles, for example particles of compound (I), can be determined by laser light diffraction. The determination can be carried out, for example, by using Beckman Coulter LS13320 laser diffraction particle size analyzer equipped with Tornado Dry Powder System using air as dispersion medium with measurement pressure 24″H₂O±2″H₂O, sample amount 10 ml, system controlled target 5% for obscuration and applying Fraunhofer optical model.

In the inhalation composition the particles of compound (I), and optionally ivabradine or N-desmethyl ivabradine or a pharmaceutically acceptable salt thereof, are suitably mixed with a suitable carrier or diluent. The amount of the active ingredient(s) is preferably from about 0.01% to about 20%, more preferably from about 0.02% to about 10%, for example from about 0.05% to about 5%, based on the total weight of the composition.

Examples of suitable solid carriers for dry powder inhalation compositions are saccharides, e.g. lactose, mannitol or glucose, having larger particle size than the active substance, for example having volume diameter in the range from about 10 μm to about 250 μm. Suitable carriers for pressurized aerosol inhalation compositions are aerosol carriers, especially non-chlorofluorocarbon based carriers, for example HFA (hydrofluoroalkane). For nebulized compositions, aqueous carriers are preferred.

Inhalation compositions may also comprise other excipients such as stabilizers, surfactants, solubilizers, flavouring, and/or preserving agents.

According to one embodiment, a single dose of inhalation composition is able to provide a plasma concentration of compound (I) of at least about 0.5 ng/ml, preferably at least about 1 ng/ml, more preferably from about 1 to about 5 ng/ml, for example from about 1 to about 2 ng/ml, within about 15 minutes, preferably within about 10 minutes, for example within about 5 minutes, from the time of administration.

An unit dose of the inhalation composition for humans comprises an amount of compound (I) which is generally in the range from about 1 μg to about 1000 μg, preferably from 10 μg to 800 μg, for example from 25 μg to 600 μg. If ivabradine or N-desmethyl ivabradine or a pharmaceutically acceptable salt thereof is also used, its amount in the unit dose of the inhalation composition is generally in the range from about 0.01 mg to about 50 mg, preferably from 0.1 mg to 30 mg, for example from 0.25 mg to 20 mg,

According to one embodiment, the pharmaceutical composition adapted for pulmonary administration, for example an inhalation composition, comprises, per weight of the composition, from about 0.02% to about 10%, for example from about 0.05% to about 5%, of compound (I), and form about 90% to about 99.98%, for example form about 95% to about 99.95%, of a carrier.

According to another embodiment, the pharmaceutical composition adapted for pulmonary administration, for example an inhalation composition, comprises, per weight of the composition, from about 0.02% to about 10%, for example from about 0.05% to about 5%, of compound (I), from about 0.02 to about 10%, for example from about 0.05 to about 5%, of ivabradine or N-desmethyl ivabradine or a pharmaceutically acceptable salt thereof and form about 80% to about 99.96%, for example form about 90% to about 99.9%, of a carrier.

The inhalation compositions may be administered using conventional delivery techniques. Dry inhalation powders may be packed, e.g. in hard gelatin capsules or a blister package to be given as single units from inhalers designed to break the capsule or blister before inhalation or directly in a reservoir of a dry powder inhaler, e.g. multidose reservoir inhalers. Aerosol compositions may be packaged to pressurized metered dose inhalers (MDI) and aqueous suspensions or solutions to nebulizer devices.

The invention is further illustrated by the following examples, which are not meant to limit the scope of the invention.

### Formulation Example 1a

Eye drop formulation

| | |
|---|---|
| Compound (I) | 0.5 mg |
| Sodium chloride (0.9%) in water | ad 1 ml |
| pH 6.1 | |

### Formulation Example 1b

Eye drop formulation

| | |
|---|---|
| Compound (I) | 0.5 mg |
| Ivabradine hydrochloride | 2.0 mg |
| Sodium chloride (0.9%) in water | ad 1 ml |
| pH 6.9 | |

### Formulation Example 2a

Intranasal drop formulation

| | |
|---|---|
| Compound (I) | 0.1 mg |
| Sodium chloride (0.9%) in water | ad 1 ml |
| pH 6.1 | |

### Formulation Example 2b

Intranasal drop formulation

| | |
|---|---|
| Compound (I) | 0.1 mg |
| Ivabradine hydrochloride | 2.0 mg |
| Sodium chloride (0.9%) in water | ad 1 ml |
| pH 6.5-6.7 | |

The above formulations can be prepared by dissolving the drug substance(s) in the carrier solution followed by filtration.

### Formulation Example 3a

Oromucosal gel formulation

| | |
|---|---|
| Compound (I) | 0.1 mg |
| Hydroxypropyl Cellulose | 150 mg |
| Propylene Glycol | 300 mg |
| Sodium Lauryl Sulphate | 10 mg |
| HCl, dilute | q.s. |
| NaOH (2M) | q.s. |
| Brilliant Blue FCF (E133) | 0.03 mg |
| water | ad 1 ml |
| pH 6.0 | |

### Formulation Example 3b

Oromucosal gel formulation

| | |
|---|---|
| Compound (I) | 0.1 mg |
| Ivabradine hydrochloride | 20 mg |
| Hydroxypropyl Cellulose | 150 mg |
| Propylene Glycol | 300 mg |
| Sodium Lauryl Sulphate | 10 mg |
| HCl, dilute | q.s. |
| NaOH (2M) | q.s. |
| Brilliant Blue FCF (E133) | 0.03 mg |
| water | ad 1 ml |
| pH 6.0 | |

### Formulation Example 3c (for Experiment 3)

Oromucosal gel formulation

| Ivabradine hydrochloride | 20 | mg |
|---|---|---|
| Hydroxypropyl Cellulose | 126 | mg |
| Propylene Glycol | 460 | mg |
| Sodium Lauryl Sulphate | 8.4 | mg |
| HCl, dilute | q.s. | |
| NaOH (2M) | q.s. | |
| Brilliant Blue FCF (E133) | 0.0252 | mg |

| | |
|---|---|
| water | ad 1 ml |
| pH 6.0 | |

The above gel formulations were prepared by adding propylene glycol, colouring agent, sodium lauryl sulphate and water in a vessel. The mixture was stirred until it was miscible and homogenous. The mixture was warmed to 50° C. Hydroxypropyl cellulose was slowly added under stirring. The gel was cooled to room temperature under gentle stirring and the drug substance(s) was added under stirring. The pH of the composition was adjusted to 6.0 by dropwise addition of HCl or NaOH solution. Clear gel was obtained after standing.

### Formulation Example 4a

Dry powder inhalation 50 μg formulation (dose weight 10 mg)

| | |
|---|---|
| Compound (I) micronized | 1 g |
| Lactose monohydrate | 199 g |

14 g of carrier lactose monohydrate is placed into a metal mixing vessel. 1 g of the micronised compound (I) is then added and the mixture is mixed for 5 min. An additional 185 g of the lactose monohydrate is then added to the mixing vessel, and the resultant mixture is tumbled for 5 min. The resultant blend is then passed through a 0.6 mm screen. The screened blend (i.e. the portion of the blend that passed through the screen) is finally reblended for 5 min. The formulation is administered from a dry powder inhalation device releasing a 10 mg unit dose of the formulation.

### Formulation Example 4b

Dry powder inhalation formulation (dose weight 10 mg)

| | |
|---|---|
| Compound (I) micronized | 1 g |
| Ivabradine hydrochloride micronized | 20 g |
| Lactose monohydrate | 179 g |

79 g of carrier lactose monohydrate is placed into a metal mixing vessel. The micronized drug substances are then added and the mixture is mixed for 5 min. An additional 100 g of the lactose monohydrate is then added to the mixing vessel, and the resultant mixture is tumbled for 5 min. The resultant blend is then passed through a 0.6 mm screen. The screened blend (i.e. the portion of the blend that passed through the screen) is finally reblended for 5 min. The formulation is administered from a dry powder inhalation device releasing a 10 mg unit dose of the formulation.

### Formulation Example 5a

Nebulized solution for inhalation 50 μg
formulation (metering volume 2.5 μl)

| | | |
|---|---|---|
| Compound (I) | 20 | mg |
| Water | 1000 | ml |

Compound (I) is dissolved in water. The solution is administered by metering a 2.5 μl unit dose of the solution from a nebulizer device.

### Formulation Example 5b

Nebulized solution for inhalation 50 μg
formulation (metering volume 2.5 μl)

| | | |
|---|---|---|
| Compound (I) | 20 | mg |
| Ivabradine hydrochloride | 400 | mg |
| Water | 1000 | ml |

Drug substances are dissolved in water. The solution is administered by metering a 2.5 μl unit dose of the solution from a nebulizer device.

Experiment 1. Bioavailability and Local Tolerance of Compound (I) Given by Ocular, Intranasal or Oromucosal Administration to Dogs.

Methods:

Bioavailability of compound (I) given by ocular (4 μg/kg), intranasal (4 μg/kg) or oromucosal dosing (5 μg/kg) was compared to 4 μg/kg intravenous (i.v.) administration in Beagle dogs.

The study was a crossover study with at least 6 days washout period between the treatment periods. The animals were fasted overnight before dosing by withdrawing the remaining feed (if any) at least 12 h before dosing. On dosing days, food was offered approximately at 4 h after dosing (after the 4 h blood sampling).

Composition of Formulation Example 1 was used for ocular administration, composition of Formulation Example 2 was used for intranasal administration and composition of Formulation Example 3 was used for oromucosal administration. Formulations and dosing are summarized in Table 1.

TABLE 1
Formulations and dosing used in the experiment.

| | | | Dose | Dose | |
|---|---|---|---|---|---|
| | Dose | Dose | Volume | Concentration | Number of |
| Period | Route | (μg/kg) | (μl/kg) | (mg/ml) | animals (n) |
| 1 | ocular | 4 | 8 | 0.5 | 6 |
| 2 | i.v. | 4 | 400 | 0.01 | 6 |
| 3 | oromucosal | 5 | 50 | 0.1 | 6 |
| 4 | intranasal | 4 | 40 | 0.1 | 6 |

Ocular and intranasal doses were divided equally to both eyes or both nostrils. Oromucosal gel formulation was administered to buccal/gingival mucosa of the dog. The intravenous dose was administered in cephalic vein in slow i.v. bolus dosed within 30 seconds.
Blood samples were collected into K₂EDTA tubes over a 48 h period after each treatment at the following time points: predose, 2 min, 5 min, 10 min, 20 min, 30 min, 45 min, 1 h, 2 h, 4 h, 6 h, 8 h, 12 h, 24 h and 48 h post administration. Aliquots of plasma were prepared and stored frozen until analysis. Compound (I) concentration in plasma was determined using Liquid chromatography-triple quadrupole mass spectrometry. The lower limit of quantification for compound (I) in dog plasma was 0.1 ng/ml.

Results:

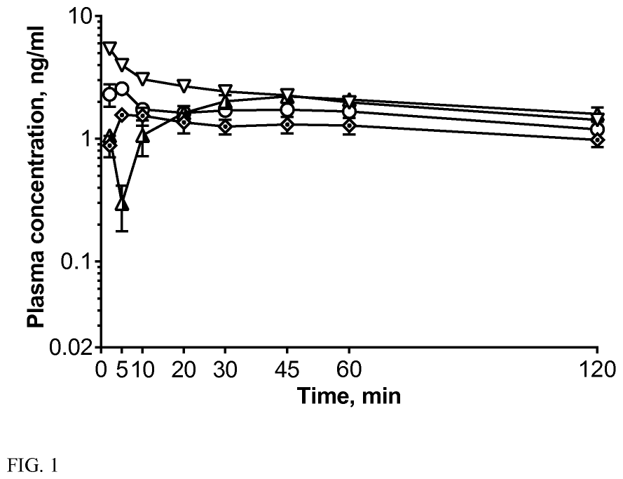

The plasma concentration vs. time curves are shown in FIG. 1 as follows:

[symbol] Eye drops, 4 μg/kg

[symbol] Intravenous, 4 gig/kg

[symbol] Oromucosal, 5 μg/kg

[symbol] Intranasal, 4 μg/kg

Compound (I) given as ocular drops, intranasal drops or oromucosal gel had a fast absorption comparable to intravenous administration, good systemic exposure and extended elimination phase. The bioavailability was 67%, 82% and 78%, for eye drops, intranasal drops and oromucosal gel, respectively. No significant irritation was seen in any administration routes.

Experiment 2. Bioavailability of Compound (I) Given by Oropharyngeal Aspiration to Rats

Methods:

Oropharyngeal aspiration administration has been developed as an alternative technique for the inhaled delivery of medicaments to the lungs and provides a means to mimic human lung exposure of medicaments by inhalation (Hulland, K. et al., American Journal of Respiratory and Critical Care Medicine 2016, 193:A5928 and Barbayianni, I. et al., Frontiers of Medicine, September 2018, Vol 5, Article 269, 1-6).

Bioavailability of compound (I) given by oropharyngeal aspiration (dosing 10 or 20 μg/kg) was compared to 10 μg/kg intravenous (i.v.) administration in Male Sprague Dawley rats.

Composition for oropharyngeal dosing was as follows:

| | |
|---|---|
| Compound (I) | 0.03 or 0.06 mg |
| Sodium chloride (0.9%) in water | ad 1 ml |

The oropharyngeal dose was administered to animals which were lightly anaesthetized by isoflurane inhalation. The animals were placed on a vertical stand the top teeth hooked over a rubber band to open the mouth. The 100 μl dose was dispensed by positive displacement pipette at the back of the throat of the animal while holding the tongue to prevent swallowing and nose held to encourage breathing through the mouth for (at least) 3 breaths. The nose released but tongue continually held for an additional couple of breaths to ensure solution deposited in the lungs and not swallowed. All animals recovered consciousness post dose.

The intravenous dose was administered as a slow bolus injection over 30 seconds via the lateral tail vein. Formulations and dosing are summarized in Table 2.

TABLE 2
Formulations and dosing used in the experiment.

| | | Dose | Dose | |
|---|---|---|---|---|
| Dose | Dose | Volume | Concentration | Number of |
| Route | (mg/kg) | (ml/kg) | (mg/ml) | animals (n) |
| oropharyngeal | 0.01 | 0.333* | 0.03 | 16 |
| oropharyngeal | 0.02 | 0.333* | 0.06 | 16 |
| i.v. | 0.01 | 2 | 0.005 | 12 |

*Assuming a nominal bodyweight of 300 g per rat

Blood samples were collected into a K₂EDTA tube such as to obtain a sample after 5 min, 15 min, 30 min, 1 h, 3 h, 5 h, 8 h, 12 h, 24 h and 48 h post administration.

Aliquots of plasma were prepared and stored frozen until analysis. Compound (I) concentration in plasma was determined using a liquid chromatography-tandem mass spectrometry (LC-MS/MS). The lower limit of quantification for compound (I) in rat plasma was 0.02 ng/ml.

Results:

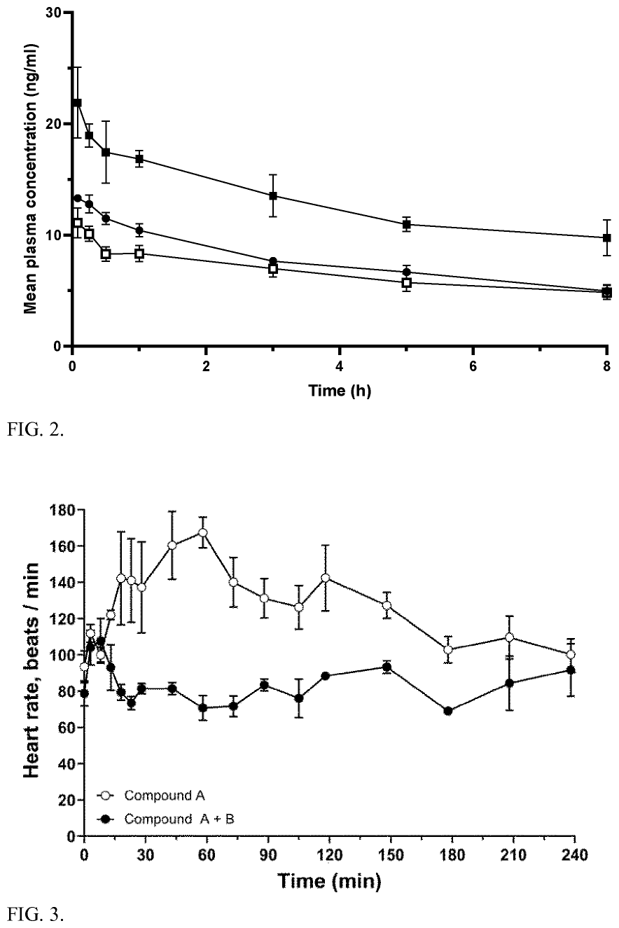

The plasma concentration vs. time curves are shown in FIG. 2 as follows:

[symbol] Intravenous bolus 0.01 mg/kg

[symbol] Oropharyngeal 0.01 mg/kg

[symbol] Oropharyngeal 0.02 mg/kg

Compound (I) given as oropharyngeal route had a fast absorption comparable to intravenous administration and good systemic exposure indicating that compound (I) is suitable for pulmonary administration by inhalation. The bioavailability of oropharyngeal administration was 94.9% and 97.1% following a dose of 0.01 and 0.02 mg/kg, respectively.

Experiment 3. Effect of Compound (I) Alone or Together with Ivabradine on the Heart Rate of Beagle Dogs by Oromucosal Administration

Methods:

Male beagle dogs (n=3) were given a dose of compound (I) using oromucosal gel according to Formulation Example 3a alone (dosing period 1) or together with a dose of ivabradine using oromucosal gel according to Formulation Example 3c (dosing period 2). The dose of compound (I) was 18.5 μg/kg and the dose of ivabradine was 1 mg/kg (calculated as ivabradine base). Each dose was divided into two sites on gingival mucosa. The washout period between the dosing periods was at least 6 days. Heart rate of the animals was assesses at baseline and post-dosing using telemetry acquisition system.

Results:

The heart rate vs. time curves are shown in FIG. 3 as follows:

A: Compound (I) 18.5 μg/kg

A+B: Compound (I) 18.5 μg/kg+ivabradine hydrochloride 1 mg/kg

It can be seen that oromucosal administration of compound (I) (“A” in the Figure) at the dose of 18.5 μg/kg increased heart rate from the baseline of 93±9 bpm to 167±8 bpm. Concurrent oromucosal administration of ivabradine hydrochloride (“B” in the Figure) at the dose of 1 mg/kg was able to completely prevent the heart rate increase induced by oromucosal administration of compound (I). Similar effect was obtained when oromucosal administration of N-desmethyl ivabradine hydrochloride at dose 1 mg/kg was used instead of ivabradine hydrochloride (data not shown).

1. A method for administration of (R)—N-[4-(1,4,5,6-tetrahydro-4-methyl-6-oxo-3-pyridazinyl)phenyl]acetamide (I) to a patient in need thereof comprising administering (R)—N-[4-(1,4,5,6-tetrahydro-4-methyl-6-oxo-3-pyridazinyl)phenyl]acetamide (I) to a patient by ocular, intranasal, oromucosal or pulmonary administration.

2. The method according to claim 1 comprising administering to the eye of the patient in need thereof an effective amount of an eye drop composition comprising (R)—N-[4-(1,4,5,6-tetrahydro-4-methyl-6-oxo-3-pyridazinyl)phenyl]acetamide (I) as an active ingredient.

3. The method according to claim 1 comprising administering to the nasal mucosa of the patient in need thereof an effective amount of an intranasal composition comprising (R)—N-[4-(1,4,5,6-tetrahydro-4-methyl-6-oxo-3-pyridazinyl)phenyl]acetamide (I) as an active ingredient.

4. The method according to claim 1 comprising administering to the oral mucosa of the patient in need thereof an effective amount of an oromucosal composition comprising (R)—N-[4-(1,4,5,6-tetrahydro-4-methyl-6-oxo-3-pyridazinyl)phenyl]acetamide (I) as an active ingredient.

5. The method according to claim 1 comprising administering by inhalation to the patient in need thereof an effective amount of an inhalation composition comprising (R)—N-[4-(1,4,5,6-tetrahydro-4-methyl-6-oxo-3-pyridazinyl)phenyl]acetamide (I) as an active ingredient.

6. A pharmaceutical composition adapted for ocular, intranasal, oromucosal or pulmonary administration comprising (R)—N-[4-(1,4,5,6-tetrahydro-4-methyl-6-oxo-3-pyridazinyl)phenyl]acetamide (I) as an active ingredient.

7. The composition according to claim 6, which is adapted for ocular administration.

8. The composition according to claim 7, which is an eye drop composition.

9. The composition according to claim 8 comprising 0.001-0.5%, per weight of the composition, of (R)—N-[4-(1,4,5,6-tetrahydro-4-methyl-6-oxo-3-pyridazinyl)phenyl]acetamide (I).

10. The composition according to claim 9 comprising at least 90%, per weight of the composition, of sterile water.

11. The composition according to claim 9 comprising 0.5-2%, per weight of the composition, of a tonicity adjusting agent.

12. The composition according to claim 11, wherein the tonicity adjusting agent is sodium chloride or mannitol.

13. The composition according to claim 9 comprising 0.1-5%, per weight of the composition, of a thickening agent.

14. The composition according to claim 13, wherein the thickening agent is polyvinylpyrrolidone, polyvinyl alcohol, polyethylene glycol, polyacrylic acid or a cellulose derivative.

15. The composition according to claim 9 having pH between 4.0-7.0.

16. The composition according to claim 6, which is adapted for intranasal administration.

17. The composition according to claim 16, which is an intranasal spray or nasal drop composition.

18. The composition according to claim 16, comprising 0.001-0.5%, per weight of the composition of (R)—N-[4-(1,4,5,6-tetrahydro-4-methyl-6-oxo-3-pyridazinyl)phenyl]acetamide (I).

19. The composition according to claim 18 comprising at least 90%, per weight of the composition, of sterile water.

20. The composition according to claim 18 comprising 0.5-2%, per weight of the composition, of a tonicity adjusting agent.

21. The composition according to claim 20, wherein the tonicity adjusting agent is sodium chloride or mannitol.

22. The composition according to claim 18 comprising 0.1-5%, per weight of the composition, of a thickening agent.

23. The composition according to claim 22, wherein the thickening agent is polyvinylpyrrolidone, polyvinyl alcohol, polyethylene glycol, polyacrylic acid or a cellulose derivative.

24. The composition according to claim 18 having pH between 4.0-7.0.

25. The composition according to claim 6, which is adapted for oromucosal administration.

26. The composition according to claim 25, which is an oromucosal spray composition, an oromucosal gel composition, a sublingual composition or a mucoadhesive oromucosal composition.

27. The composition according to claim 25 or 26, comprising 0.002-0.5%, per weight of the composition of (R)—N-[4-(1,4,5,6-tetrahydro-4-methyl-6-oxo-3-pyridazinyl)phenyl]acetamide (I).

28. The composition according to claim 25, which is a semisolid oromucosal gel.

29. The composition according to claim 28, which comprises a gelling agent, water-miscible organic co-solvent and water.

30. The composition according to claim 6, which is adapted for pulmonary administration.

31. The composition according to claim 30, which is in the form of a dry powder inhalation composition, a pressurized aerosol inhalation composition or a nebulized inhalation composition.

32. The composition according to claim 30 or 31, comprising 0.02-10%, per weight of the composition, of (R)—N-[4-(1,4,5,6-tetrahydro-4-methyl-6-oxo-3-pyridazinyl)phenyl]acetamide (I).

33. The composition according to claim 32, comprising particles of (R)—N-[4-(1,4,5,6-tetrahydro-4-methyl-6-oxo-3-pyridazinyl)phenyl]acetamide (I) having volume median diameter between 0.5 μm-10 μm.

34. The composition according to claim 33, which is a dry powder inhalation composition further comprising particles of a carrier.

35. The composition according to claim 34, wherein the carrier is lactose, mannitol or glucose.

36. The composition according to claim 35, wherein the volume diameter of the carrier particles is from about 10 μm to about 250 μm.

37. An inhaler or nebulizer device comprising a composition of (R)—N-[4-(1,4,5,6-tetrahydro-4-methyl-6-oxo-3-pyridazinyl)phenyl]acetamide (I) adapted for pulmonary administration.

38-39. (canceled)

40. A method for administration of a composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof, to a patient in need thereof comprising administering said composition to a patient by ocular, intranasal, oromucosal or pulmonary administration.

41-43. (canceled)

44. A pharmaceutical composition comprising (R)—N-[4-(1,4,5,6-tetrahydro-4-methyl-6-oxo-3-pyridazinyl)phenyl]acetamide (I) in combination with ivabradine or N-desmethyl ivabradine or a pharmaceutically acceptable salt thereof.
